(19) United States
(12) Patent Application Publication
(10) Pub. No.: US 2024/0240221 A1
(43) Pub. Date: Jul. 18, 2024

(54) METHOD FOR THE HYDROXYLATION OF STEROIDS

(71) Applicant: PHARMAZELL GMBH, Raubling (DE)

(72) Inventors: Nicole Staunig, Vasoldsberg (AT); Kai Oliver Donsbach, Traunstein (DE)

(21) Appl. No.: 17/908,507

(22) PCT Filed: Mar. 5, 2021
(86) PCT No.: PCT/EP2021/055617

§ 371 Date: Aug. 31, 2022

(30) Foreign Application Priority Data
Mar. 6, 2020 (EP) 20161536.6

(51) Int. Cl. C12P 33/06

(52) U.S. Cl. CPC C12P 33/06

(57) ABSTRACT

The present invention relates to an enzme and method for the hydroxylation of a 7-deoxysteroid having the general formula (I)

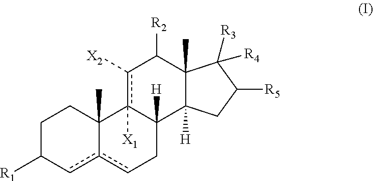

at position 7 to a steroid having the general formula (II)

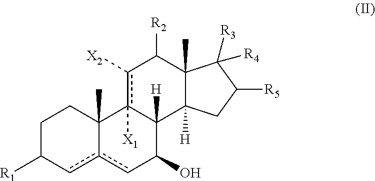

### CROSS-REFERENCE TO RELATED APPLICATIONS

This Application is a 371 of International Patent Application No. PCT/EP2021/055617, filed Mar. 5, 2021, which claims the benefit of European Patent Application No. 20161536.6, filed Mar. 6, 2020, which are incorporated herein by reference in their entireties.

### SEQUENCE LISTING

The instant application contains a Sequence Listing that has been submitted electronically in ASCII format and is hereby incorporated by reference in its entirety. Said ASCII copy, created on Jan. 27, 2023, is named “16785-324_2023-01-30_Sequence-Listing” and is 22.6 kb in size.

### TECHNICAL FIELD

The present invention relates to means and methods for the hydroxylation of steroids.

### BACKGROUND OF THE INVENTION

3,7,12-Trihydroxylated bile acids, such as, e.g., cholic acid (3a,7α,12a-trihydroxy-50-cholanic acid) or ursocholic acid (3a,70,12α-trihydroxy-50-cholanic acid), are industrially important chemicals, among other things as starting materials for the manufacture of ursodeoxycholic acid (UDCA). Ursodeoxycholic acid is used, among other things, as a medicament for dissolving minor X-ray negative gallstones as well as for treating the liver diseases primary ciliary cirrhosis and primary sclerosing cholangitis.

The industrially most important source of 3,7,12-trihydroxylated bile acids is biliary fluid from gallbladders, which accumulate as slaughterhouse waste in meat production. Besides other animal species, the bile of cattle is often used. There is no industrially relevant total synthesis for 3,7,12-trihydroxylated bile acids. Since the production of bile acids is linked to another product (meat), the response to increased demands can only be very limited. For this reason, it is of great interest to use the raw material bile as efficiently as possible.

Since bile is an aqueous mixture of bile acids, lipids, cholesterol and other substances, the separation of the components during the extraction of bile acids is of particular importance. The bile acids, in turn, also constitute a mixture the components of which differ in the number and position of the hydroxyl groups. In addition to cholic acid, bovine bile also contains a significant proportion of deoxycholic acid, which differs from cholic acid in that the OH group is missing at position 7 (3a,12α-dihydroxy-5β-cholanic acid). Deoxycholic acid has a much lower commercial value than 3,7,12-trihydroxylated bile acids. Therefore, there is an industrial interest in converting deoxycholic acid into a 3,7,12-trihydroxylated bile acid by selectively introducing a hydroxyl group at position 7.

During hydroxylations, an oxygen atom is formally introduced into a (non-activated) C—H bond in an oxidation reaction. In organic chemistry, these are reactions that are very difficult to perform. OH groups are frequently introduced through detours, e.g., by adding water at a C=C double bond. The selective hydroxylation at a specific position of a complex molecule (such as, e.g., a bile acid) is problematic, since several chemically (almost) equivalent C—H bonds are present.

It is known that certain actinobacteria and filamentous fungi can catalyze the regio-and stereospecific hydroxylation of lithocholic acid (“LCA”) at position 73 to ursodeoxycholic acid (“UDCA”) (Kollerov et al., Steroids, 78 (3): 370-378 (2013); Tonin et al. (Beilstein Journal of Organic Chemistry, 14: 470-483 (2018)).

In addition, the hydroxylation of deoxycholic acid at position 7 by filamentous fungi (Kollerov et al., Steroids, 107: 20-29 (2016)) and actinobacteria (Deshcherevskaya et al., Journal of Molecular Catalysis B: Enzymatic, 133: p.157-p.165 (2016)) has been described.

It is an object of the present invention to provide means and methods of hydroxylating steroids, such as bile acids and derivatives thereof, which have a hydrogen and no hydroxyl group at position 7, specifically at this point.

### SUMMARY OF THE INVENTION

The object according to the invention is achieved by using cytochrome P450 or a functional fragment thereof for the hydroxylation of a 7-deoxysteroid having the general formula (I)

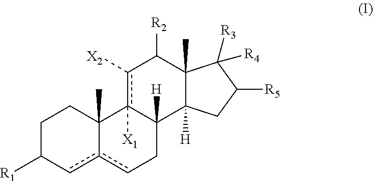

at position 7 to a steroid having the general formula (II)

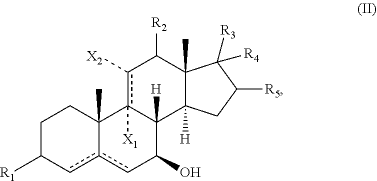

wherein
X₁ and X₂ are independently H, Cl, F, Br, I, CF₃, a C₁ to C₆ alkyl radical, OH, a C₁ to C₆ alkoxy radical, CN, NO₂, N(R₆)₂, an epoxy group, CHO or a CO₂R₆ radical, wherein
   R₆ is —C(O)H, —C(O)CH₃, —C(O)CH₂CH₃, —C(O)(CH₂)₂CH₃, —C(O)CH(CH₃)₂, —C(O)(CH₂)₃CH₃, —C(O)CH(CH₃)CH₂CH₃, —C(O)CH₂CH₂(CH₃)₂, —C(O)C(CH₃)₃, —C(O)Ph, —C(O)CH₂Ph,
R₁ and R₂ are independently H, OH, OR₂ or O, wherein
   R₇ is —C(O)H, —C(O)CH₃, —C(O)CH₂CH₃, —C(O)(CH₂)₂CH₃, —C(O)CH(CH₃)₂, —C(O)(CH₂)₃CH₃, —C(O)CH(CH₃)CH₂CH₃, —C(O)CH₃CH₂(CH₃)₂, —C(O)C(CH₃)₃, —C(O)Ph, —C(O)CH₂Ph,
R₃ is H, OH, OR₈, a C₁ to C₁₀ alkyl radical, a C₁ to C₁₀ alkenyl radical, —CHO, —C(O)(CH₃), —C(O)(CH₂OH), —CH(CH₃)C(O)CH₃, —CH(CH₃)((CH₂)₂CO₂R₉) or —CH(CH₃)((CH₂)₂CONHR₉), wherein
   R₈ is —C(O)H, —C(O)CH₃, —C(O)CH₂CH₃, —C(O)(CH₃)CH₃ —C(O)CH(CI3)₂, —C(O)(CH₂)₃CH₃, —C(O)CH(CH₃)CH₂CH₃, —C(O)CH₂CH₂(CH₃)₂, —C(O)C(CH₃)₃, —C(O)Ph or —C(O)CH₂Ph, and
   R₉ is —CH₃, —CH₂COOH, —CH₂CH₃, —CH(CH₃)₂, —(CH₂)₂CH₃, —(CH₂)₂SO₃H, C(CH₃)₃, —(CH₂)₃CH₃, —CH(CH₃)CH₂CH₃, —CH₂CH₂(CH₃)₂, an aryl group or an alkylaryl group,
R₄ is H, OH, or —OR₁₀, wherein
   R₁₀ is —C(O)H, —C(O)CH₃, —C(O)CH₂CH₃, —C(O)(CH₂)₂CH₃, —C(O)CH(CH₃)₂. —C(O)(CH₂)₃CH₃, —C(O)CH(CH₃)CH₂CH₃, —C(O)CH₂CH₂(CH₃)₂, —C(O)C(CH₃)₃, —C(O)Ph or —C(O)CH₂Ph, and
R₅ is H, CF₃, a C₁ to C₆ alkyl radical, a C₁ to C₆ alkenyl radical, OH, O, or a C₁ to C₆ alkoxy radical, wherein the dashed line denotes an optional double bond, with the proviso that the B ring has no double bond if the A ring has a C₄-C₅ double bond, and the C ring has no double bond if X₁ and X₂ form an epoxy group,
characterized in that the cytochrome P450 enzyme comprises an amino acid sequence which is at least 80%, preferably at least 90%, in particular 100%, identical to the amino acid sequence SEQ ID No. 1 or 2.

Surprisingly, it has been shown that cytochrome P450 and functional fragments thereof are capable of hydroxylating steroids such as cholic acid and, respectively, derivatives thereof having the formula (I) at position 7.

A further aspect of the present invention relates to a method of preparing a steroid, preferably a cholic acid, or a derivative thereof having the general formula (II) as defined above, comprising the step of converting a 7-deoxysteroid, preferably a 7-deoxycholic acid, or a derivative thereof having the general formula (I) with the cytochrome P450 according to the invention or a functional variant thereof.

### DESCRIPTION OF THE EMBODIMENTS

Cytochrome P450 and functional variants thereof are surprisingly capable of selectively hydroxylating 7-deoxysteroids, such as, e.g., 7-deoxycholic acid, and derivatives thereof at position 7.

According to the present invention, the cytochrome P450 enzyme comprises an amino acid sequence which is at least 80%, preferably at least 90%, in particular 100%, identical to the amino acid sequence SEQ ID No. 1 or 2.

Cytochromes P450 catalyze monooxygenase reactions of a large number of endogenous as well as exogenous substrates. They are involved, among other things, in the metabolism of steroids, eicosanoids, fatty acids and bile acids as well as of exogenous substrates such as drugs, insecticides and chemical carcinogens.

Cytochromes P450 according to the present invention can be used, for example, from bacteria such as actinobacteria, in particular, for example, from the genus Streptomyces. In this case, the sequences can be isolated, for example, from genomic DNA or a cDNA library using known techniques.

The cytochromes P450 according to the present invention and, respectively, their functional variants can optionally be present in their original organism or can be isolated therefrom, or they are expressed recombinantly or produced synthetically. Recombinantly expressed polypeptides are preferably used according to the invention.

Various established microorganisms can be used for the recombinant expression of enzymes according to the present invention, such as, e.g., Escherichia coli (E. coli), Bacillus subtilis, Saccharomyces cerevisiae or Pichia pastoris. Appropriate protocols in this regard are described in detail in the relevant specialist literature or are known to a person skilled in the art.

According to the present invention, enzymes/polypeptides are preferably used as proteins recombinantly overexpressed in E. coli, with the corresponding cell lysates preferably being used either without further processing/purification or after relatively simple processing steps (e.g., centrifugation, precipitation, concentration or lyophilization). After the recombinant overexpression of the enzymes used, E. coli cells can alternatively also be used in the reaction directly without cell disintegration or, for example, after a freezing/thawing cycle. Suitable expression plasmids are known to a person skilled in the art and can often be purchased commercially.

“Functional variants” of cytochrome P450 can be fragments or mutational variants of cytochrome P450, wherein fragments of cytochrome P450 can also be referred to as “functional fragments”. “Functional variants” of cytochrome P450 are capable of catalyzing the same reaction as the protein from which they have been derived. Whether a variant is functional, i.e., whether it catalyzes the same reaction as the protein from which it is derived, can be determined by establishing that the variant catalyzes the same reaction. For this purpose, there are established methods in the prior art or, respectively, those that are described herein. The conversion rates of substrates by the functional variants according to the invention can deviate from the conversion rates of the cytochrome P450 from which they have been derived.

“Derivatives of 7-deoxysteroids” comprise compounds derived from 7-deoxysteroids and having a wide variety of modifications, with one or several modifications at positions 3, 12 and 17 of the 7-deoxysteroid being particularly preferred. Such modifications preferably comprise substitutions as defined above.

According to a preferred embodiment of the present invention, X₁, X₂, R₄ and R₅ are H and
R₁ und R₂ are independently H, OH, OR₈ or O, wherein
   R₈ is —C(O)H, —C(O)CH₃, —C(O)CH₂CH₃, —C(O)(CH₁)₁CH₃, —C(O)CH(C₁—H₃)₂, —C(O)(CH₂)₃CH₃. —C(O)CH(CH₃)CH₂CH₃, —C(O)CH₂CH₂(CH₃)₂, —C(O)C(CH₃)₃, —C(O)Ph, —C(O)CH₂Ph,
R₃ is a C₁ to C₁₀ alkyl radical, a C₁ to C₁₀ alkylen radical, —CH(CH₃)((CH₂)₂CO₂R₉) or —CH(CH₃)((CH₂)₂CONHR₉), wherein
   R₉ is —CH₃, —CH₂C₀OH, —C-2CH₃, —CH(C₁-13)₂, —(C₁-12)₂C₁₁₃, —(CH₂)₂SO₃H, C(CH₃)₃, —(CH₂)₃CH₃, —CH(CH₃)CH₂CH₃, —CH₂CH₂(CH₃)₂, an aryl group or an alkylaryl group.

According to a further preferred embodiment of the present invention, the aryl group is selected from the group consisting of a phenyl radical, a phenyl radical substituted with F, Cl, Br, NO₂ or CH₃ and a heteroaryl.

According to yet another preferred embodiment of the present invention, the alkylaryl group is selected from the group consisting of a benzyl group, a halogenated benzyl group, wherein the halogen is F, C₁ or Br, and a benzyl group substituted with NO₂.

According to a preferred embodiment of the present invention, R₁ is OH, R₂ is 0 or OH, R₃ is CH(CH₃)((CH₂)₂CO₂R₅), R₄ is H, and R₅ is H.

According to another preferred embodiment of the present invention, the 7-deoxysteroid having the general formula (II) is selected from the group consisting of 3α,12α-dihydroxy-5β-cholane-24-acid, 3α,12β-dihydroxy-5β-cholane-24-acid, 3β,12α-dihydroxy-5β-cholane-24-acid, 3β,12β-dihydroxy-5β-cholane-24-acid, 3β-hydroxy-12-keto-5β-cholane-24-acid, 3-keto,12β-hydroxy-5β-cholane-24-acid, 3-keto,12α-hydroxy-5β-cholane-24-acid, 3α-hydroxy-5β-cholane-24-acid, 3-keto-5β-cholane-24-acid, 30-hydroxy-5β-cholane-24-acid and esters of the respective acid.

The cytochrome P450 hydroxylase used, according to the invention, for the hydroxylation of 7-deoxysteroids and derivatives thereof having the general formula (I) to a steroid or a derivative thereof having the general formula (II) comprises an amino acid sequence which is at least 80%, preferably at least 85%, more preferably at least 90%, more preferably at least 95%, more preferably at least 96%, more preferably at least 97%, more preferably at least 98%, more preferably at least 99%, in particular 100%, identical to the amino acid sequence SEQ ID No. 1 or 2.

SEQ ID No. 1:
MLTTAETTSIAYPFNTAEGLALSERYEEARNRTGLLRVRMPYGEPAWLV
TRYADARLVLGDRRFSRAEALHHDEPRQSEGRPDSGILTMDPPDHTRLR
TLVAKAFTVHQVEKLRPWVRQLTHDLLDDLEAAGPPADLVDRYALPIPV
GVICAMLGVPQEDRPKFRVWSDAALSTSSLSAEQFARNTDELRAYMAGL
IEDHRRTPRDDIMTSLIEARDAGDRLSELELVDLCVGILVAGHETTATQ
IPNFVLTLLEHPDQLRRLREDPALIQGAVEELLRFVPLGVGAAQARYAT
EDIEVGGTLVRSGEPVLVAVGSANRDALRFDEPGVLNVARPTTQHLGFG
HGVHHCLGAPLARLELQEALGALITRFPGLRLAGDIEWKDRMLVRGPRV
MPIGW
SEQ ID No. 2:
MPYGEPAWLVTRYADARLVLGDRRFSRAEALHHDEPRQSEGRRDSGILT
MDPPDHTRLRTLVAKAFTVHQVEKLRPWVRQLTHDLLDDLEAAGPPADL
VDRYALPIPVGVICAMLGVPQEDRPKFRVWSDAALSTSSLSAEQFARNT
DELRAYMAGLIEDHRRTPRDDIMTSLIEARDAGDRLSELELVDLCVGIL
VAGHETTATQIPNFVLTLLEHPDQLRRLREDPALIQGAVEELLRFVPLG
VGAAQARYATEDIEVGGTLVRSGEPVLVAVGSANRDALRFDEPGVLNVA
RPTTQHLGFGHGVHHCLGAPLARLELQEALGALITRFPGLRLAGDIEWK
DRMLVRGPRVMPIGW

Amino acid sequences SEQ ID Nos. 1 and 2 are preferable encoded by nucleic acid sequences SEQ ID Nos. 3 and 4, with nucleic acid sequences SEQ ID Nos. 5 and 6 being optimized for expression in E. coli.

SEQ ID No. 3:
ATGTTGACCACAGCCGAGACGACATCCATCGCCTATCCCTTCAACACCGC
CGAAGGGCTGGCGCTCAGCGAGCGTTACGAAGAGGCCAGGAACCGCACCG
GACTGCTCCGGGTGCGGATGCCCTACGGTGAGCCCGCCTGGCTGGTCACG
CGGTACGCCGACGCCCGGCTGGTGCTCGGCGACCGGCGCTTCAGCCGTGC
GGAGGCGCTCCACCACGACGAGCCGCGGCAGTCCGAAGGCCGGCGCGACA
GCGGCATCCTGACCATGGACCCGCCCGACCACACCCGGCTGCGCACCCTC
GTCGCCAAGGCGTTCACCGTCCACCAGGTGGAGAAACTCCGCCCCTGGGT
ACGCCAGTTGACCCATGACCTGCTCGACGACCTCGAGGCCGCCGGGCCGC
CCGCCGATCTGGTGGACCGCTACGCCCTGCCCATTCCGGTCGGCGTCATC
TGCGCCATGCTCGGCGTCCCGCAGGAGGACCGGCCCAAGTTCCGGGTCTG
GAGCGACGCCGCGCTGTCCACCAGCTCGCTGAGCGCCGAGCAGTTCGCCC
GTAACACCGACGAGCTGCGCGCCTACATGGCCGGGCTGATCGAGGACCAC
CGCAGGACCCCGCGGGACGACATCATGACCTCGCTGATCGAGGCGCGGGA
CGCGGGCGACCGGCTGTCCGAGCTGGAACTCGTCGATCTGTGCGTGGGCA
TCCTGGTGGCCGGGCACGAGACCACCGCCACCCAGATCCCCAACTTCGTG
CTGACGCTGCTGGAGCACCCGGACCAGCTGCGCCGGCTGCGCGAGGACCC
CGCCCTGATCCAGGGCGCCGTCGAGGAGCTGCTGCGCTTCGTCCCGCTGG
GCGTGGGCGCCGCCCAGGCCCGTTACGCCACCGAGGACATCGAGGTGGGC
GGCACGCTGGTGCGCAGCGGGGAGCCGGTGCTGGTCGCCGTCGGCTCGGC
CAACCGCGACGCGCTGCGCTTCGACGAACCGGGCGTGCTCAACGTCGCCC
GCCCCACCACCCAGCACCTCGGCTTCGGCCACGGTGTGCACCACTGCCTG
GGCGCGCCCCTGGCCCGTCTGGAGCTCCAGGAGGCGCTCGGCGCGCTGAT
CACGCGCTTCCCGGGCCTGCGGCTGGCCGGGGACATCGAGTGGAAGGACC
GCATGCTGGTCCGCGGGCCCCGTGTCATGCCCATCGGGTGGTGA
SEQ ID No. 4:
ATGCCCTACGGTGAGCCCGCCTGGCTGGTCACGCGGTACGCCGACGCCCG
GCTGGTGCTCGGCGACCGGCGCTTCAGCCGTGCGGAGGCGCTCCACCACG
ACGAGCCGCGGCAGTCCGAAGGCCGGCGCGACAGCGGCATCCTGACCATG
GACCCGCCCGACCACACCCGGCTGCGCACCCTCGTCGCCAAGGCGTTCAC
CGTCCACCAGGTGGAGAAACTCCGCCCCTGGGTACGCCAGTTGACCCATG
ACCTGCTCGACGACCTCGAGGCCGCCGGGCCGCCCGCCGATCTGGTGGAC
CGCTACGCCCTGCCCATTCCGGTCGGCGTCATCTGCGCCATGCTCGGCGT
CCCGCAGGAGGACCGGCCCAAGTTCCGGGTCTGGAGCGACGCCGCGCTGT
CCACCAGCTCGCTGAGCGCCGAGCAGTTCGCCCGTAACACCGACGAGCTG
CGCGCCTACATGGCCGGGCTGATCGAGGACCACCGCAGGACCCCGCGGGA
CGACATCATGACCTCGCTGATCGAGGCGCGGGACGCGGGCGACCGGCTGT
CCGAGCTGGAACTCGTCGATCTGTGCGTGGGCATCCTGGTGGCCGGGCAC
GAGACCACCGCCACCCAGATCCCCAACTTCGTGCTGACGCTGCTGGAGCA
CCCGGACCAGCTGCGCCGGCTGCGCGAGGACCCCGCCCTGATCCAGGGCG
CCGTCGAGGAGCTGCTGCGCTTCGTCCCGCTGGGCGTGGGCGCCGCCCAG
GCCCGTTACGCCACCGAGGACATCGAGGTGGGCGGCACGCTGGTGCGCAG
CGGGGAGCCGGTGCTGGTCGCCGTCGGCTCGGCCAACCGCGACGCGCTGC
GCTTCGACGAACCGGGCGTGCTCAACGTCGCCCGCCCCACCACCCAGCAC
CTCGGCTTCGGCCACGGTGTGCACCACTGCCTGGGCGCGCCCCTGGCCCG
TCTGGAGCTCCAGGAGGCGCTCGGCGCGCTGATCACGCGCTTCCCGGGCC
TGCGGCTGGCCGGGGACATCGAGTGGAAGGACCGCATGCTGGTCCGCGGG
CCCCGTGTCATGCCCATCGGGTGGTGA
SEQ ID No. 5:
ATGCTGACCACCGCAGAAACCACCAGTATTGCATATCCGTTTAATACCGC
AGAAGGTCTGGCACTGAGCGAACGTTATGAAGAAGCACGTAATCGTACCG
GTCTGCTGCGTGTTCGTATGCCGTATGGTGAACCGGCATGGCTGGTTACC
CGTTATGCAGATGCCCGTCTGGTTCTGGGTGATCGTCGTTTTAGCCGTGC
CGAAGCACTGCATCACGATGAACCGCGTCAGAGCGAAGGTCGTCGTGATA
GCGGTATTCTGACCATGGATCCGCCTGATCATACCCGTCTGCGTACCCTG
GTTGCAAAAGCATTTACCGTTCATCAGGTTGAAAAACTGCGTCCGTGGGT
TCGCCAGCTGACCCATGATCTGCTGGATGATCTGGAAGCAGCAGGTCCGC
CTGCAGATCTGGTTGATCGTTATGCACTGCCGATTCCGGTTGGTGTTATT
TGTGCAATGCTGGGTGTTCCGCAAGAAGATCGTCCTAAATTTCGTGTTTG
GAGTGATGCAGCACTGAGCACCAGCAGCCTGAGCGCAGAACAGTTTGCAC
GTAATACCGATGAACTGCGTGCATATATGGCAGGTCTGATTGAAGATCAT
CGTCGTACACCGCGTGATGATATTATGACCAGCCTGATCGAAGCACGTGA
TGCCGGTGATCGCCTGAGTGAACTGGAACTGGTGGATCTGTGTGTTGGTA
TTCTGGTTGCAGGTCATGAAACCACCGCAACCCAGATTCCGAATTTTGTT
CTGACCCTGCTGGAACATCCGGATCAGCTGCGTCGTCTGCGTGAAGATCC
GGCACTGATTCAGGGTGCAGTTGAAGAACTGCTGCGTTTTGTTCCGCTGG
GTGTGGGTGCAGCACAGGCACGTTATGCAACCGAAGATATTGAAGTTGGT
GGCACCCTGGTTCGTAGTGGCGAACCGGTGCTGGTTGCCGTTGGTAGCGC
AAACCGTGATGCACTGCGCTTTGATGAACCGGGTGTTCTGAATGTTGCAC
GTCCGACCACACAGCATCTGGGTTTTGGTCATGGTGTTCATCATTGTCTG
GGTGCACCGCTGGCACGTCTGGAACTGCAAGAAGCACTGGGAGCACTGAT
TACCCGTTTTCCGGGTCTGCGTCTGGCAGGCGATATTGAATGGAAAGATC
GTATGCTGGTTCGTGGTCCGCGTGTTATGCCGATTGGTTGGTAA
SEQ ID No. 6:
ATGGTGAACCGGCATGGCTGGTTACCCGTTATGCAGATGCCCGTCTGGTT
CTGGGTGATCGTCGTTTTAGCCGTGCCGAAGCACTGCATCACGATGAACC
GCGTCAGAGCGAAGGTCGTCGTGATAGCGGTATTCTGACCATGGATCCGC
CTGATCATACCCGTCTGCGTACCCTGGTTGCAAAAGCATTTACCGTTCAT
CAGGTTGAAAAACTGCGTCCGTGGGTTCGCCAGCTGACCCATGATCTGCT
GGATGATCTGGAAGCAGCAGGTCCGCCTGCAGATCTGGTTGATCGTTATG
CACTGCCGATTCCGGTTGGTGTTATTTGTGCAATGCTGGGTGTTCCGCAA
GAAGATCGTCCTAAATTTCGTGTTTGGAGTGATGCAGCACTGAGCACCAG
CAGCCTGAGCGCAGAACAGTTTGCACGTAATACCGATGAACTGCGTGCAT
ATATGGCAGGTCTGATTGAAGATCATCGTCGTACACCGCGTGATGATATT
ATGACCAGCCTGATCGAAGCACGTGATGCCGGTGATCGCCTGAGTGAACT
GGAACTGGTGGATCTGTGTGTTGGTATTCTGGTTGCAGGTCATGAAACCA
CCGCAACCCAGATTCCGAATTTTGTTCTGACCCTGCTGGAACATCCGGAT
CAGCTGCGTCGTCTGCGTGAAGATCCGGCACTGATTCAGGGTGCAGTTGA
AGAACTGCTGCGTTTTGTTCCGCTGGGTGTGGGTGCAGCACAGGCACGTT
ATGCAACCGAAGATATTGAAGTTGGTGGCACCCTGGTTCGTAGTGGCGAA
CCGGTGCTGGTTGCCGTTGGTAGCGCAAACCGTGATGCACTGCGCTTTGA
TGAACCGGGTGTTCTGAATGTTGCACGTCCGACCACACAGCATCTGGGTT
TTGGTCATGGTGTTCATCATTGTCTGGGTGCACCGCTGGCACGTCTGGAA
CTGCAAGAAGCACTGGGAGCACTGATTACCCGTTTTCCGGGTCTGCGTCT
GGCAGGCGATATTGAATGGAAAGATCGTATGCTGGTTCGTGGTCCGCGTG
TTATGCCGATTGGTTGGTAA

“Identical” as used herein means that two or more amino acid sequences, when superimposed on one another, may have a certain “identity” (matching amino acid residues at identical positions) to one another. “Identity” is defined in this invention as the percentage of amino acids of eligible amino acid sequences that are identical to the amino acids of the starting sequence, namely after the alignment of the two sequences and the introduction of gaps, if necessary, in order to achieve the maximum percentual sequence identity as generated by the “protein BLAST” program (blastp; Altschul et al., J. Mol. Biol. (1997) 215:403-410; http://blast.ncbi.nlm.nih.gov/Blast.cgi; commonly referred to herein as “BLAST”), with all variable parameters set to default values. Herein, the algorithm “blastp (protein-protein-BLAST)” is used with the following parameters: “expect threshold”: 0.05; “word size”: 6; matrix: BLOSUM62; “gap costs”: “Existence” 11, “Extension” 1; conditional compositional score matrix adjustment; no filter and no mask. A percentage (%) value for the amino acid sequence identity is determined by the number of matching identical nucleotides divided by the sequence length for which the identity in percent is recorded.

A further aspect of the present invention relates to a method of preparing a steroid or a derivative thereof having the general formula (II)

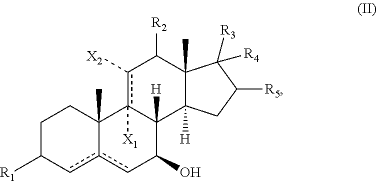

wherein
X₁ and X₂ are independently H, Cl, F, Br, I, CF₃, a C₁ to C₆ alkyl radical, OH, a C₁ to C₆ alkoxy radical, CN, NO₂, N(R₆)₂, an epoxy group, CHO or a CO₂R₆ radical, wherein
   R₆ is —C(O)H, —C(O)CH₃, —C(O)CH₂CH₃, —C(O)(CH₂)₂CH₃, —C(O)CH(CH₃)₂,—C(O)(CH₂)₃CH₃, —C(O)CH(CH₃)CH₂CH₃, —C(O)CH₂CH₂(CH₃)₂, —C(O)C(CH₃)₃, —C(O)Ph, —C(O)CH-₂Ph, R₁ and R₂ are independently H, OH, OR₇ or O, wherein
   R₇ is —C(O)H, —C(O)CH₃, —C(O)CH₂CH₃, —C(O)(CH₂)₂CH₃, —C(O)CH(CH₃)₂, —C(O)(CH₂)₃CH₃, —(O)CH—(CH₃,CO)CH₂CH₃, —C(O)CH(CH₂(CH₃)₂, —C(O)C(CH₃), —C(O)Ph, —C(O)CH₂Ph,
R₃ is H, OH, OR₈, a C₁ to C₁₀ alkyl radical, a C₁ to C₁₀ alkenyl radical, —CHO, —C(O)(CH₃), —C(O)(CH₂OH)), —CH(CH₃)C(O)C₁₃, —CH(CH₃)((CH₂)₂CO₂R₉) or —CH(CH₃)((CH₂)₂CONHR₉), wherein
   R₈ is —C(O)H, —C(O)CH₃, —C(O)CH₂CH₃, —C(O)(CH₂)₂CH₃, —C(O)CH(CH₃)₂, —C(O)(CH₂)₃CH₃, —C(O)CH(CH₃)CH₂CH₃, —C(O)CH₂CH₂(CH₃)₂, —C(O)C(CH₃)₃, —C(O)Ph or —C(O)CH₂Ph, and
   R₉ is —CH₃, —CH₂COOH, —CH₂CH₃, —CH(CH₃)₂, —(CH₂)₂CH₃, —(CH₂)₂S03H, C(CH₃)₃, —(CH₂)₃CH₃, —CH(CH₃)CH₂CH₃, —CH₂CH₂(CH₃)₂, an aryl group or an alkylaryl group, R₄ is H, OH, or —OR₁₀, wherein
   R₁₀ is —C(O)H, —C(O)CH₃, —C(O)CH₂CH₃, —C(O)(CH₂)₂CH₃, —C(O)CH(CH₃)₂, —C(O)(CH₂)₃C₁-3, —C(O)CH(CH₃)CH₂CH₃, —C(O)CH₂CH₂(CH₃)₂, —C(O)C(CH₃)₃, —C(O)Ph or —C(O)CH₂Ph, and
R₅ is H, CF₃, a C₁ to C₆ alkyl radical, a C₁ to C₆ alkenyl radical, OH, O, or a C₁ to C₆ alkoxy radical, wherein the dashed line denotes an optional double bond, with the proviso that the B ring has no double bond if the A ring has a C₄-C₅ double bond, and the C ring has no double bond if X₁ and X₂ form an epoxy group,
comprising the step of converting a 7-deoxysteroid or a derivative thereof having the general formula (I)

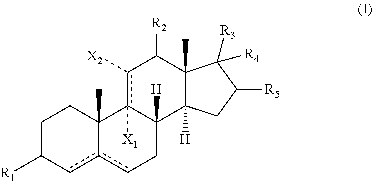

with cytochrome P450 or a functional variant thereof, characterized in that the cytochrome P450 enzyme comprises an amino acid sequence which is at least 80%, preferably at least 90%, in particular 100%, identical to the amino acid sequence SEQ ID No. 1 or 2.

Using the method according to the invention, 7-deoxysteroids or, respectively, derivatives thereof having the general formula (I) can be converted with cytochrome P450 according to the invention or a functional variant thereof to steroids or, respectively, derivatives thereof having the general formula (II).

In order to support the redox reaction of cytochrome P450 according to the invention or, respectively, its functional variants thereof, it is advantageous to carry out the method according to the invention in the presence of a reducing agent. NAD(P)H, flavins or ferredoxins can be used as reducing agents. For example, if the redox cofactors NAD(P)+ and/or NAD(P)H are used, it is advantageous to use them at a concentration of 0.001 mM and 10 mM, more preferably between 0.05 mM and 1 mM, in a reaction mixture.

The method according to the invention is preferably carried out in the presence of redox partners for cytochrome P450. Redox partners are understood to be proteins of the ferredoxin and ferredoxin reductase classes, which are advantageous for the function of cytochrome P450 according to the present invention. A possible pair of redox partners preferably comprises putidaredoxin and putidaredoxin reductase from Pseudomonas putida. Moreover, a person skilled in the art is able to identify further ferredoxin proteins and ferredoxin reductases which are potential redox partners for the cytochrome P450 according to the invention. Suitability as a redox partner can be verified in a functional assay, as described, for example, in Examples 3 to 5. The putidaredoxin used in these examples and/or the putidaredoxin reductase used therein can be replaced by possible alternative proteins or enzymes, respectively. If sufficient formation of the desired product (e.g., ursocholic acid) is observed, the tested redox partners can be regarded as functional alternatives to putidaredoxin and/or putidaredoxin reductase.

According to a particularly preferred embodiment of the present invention, the ferredoxin used in the method according to the invention comprises an amino acid sequence which is at least 80%, preferably at least 85%, more preferably at least 90%, more preferably at least 95%, more preferably at least 96%, more preferably at least 97%, more preferably at least 98%, more preferably at least 99%, in particular 100%, identical to the amino acid sequence SEQ ID No. 7, wherein X is a methionine residue or is not an amino acid.

SEQ ID No. 7:
XSKVVYVSHDGTRRELDVADGVSLMQAAVSNGIYDIVGDCGGSASCAT
CHVYVNEAFTDKVPAANEREIGMLECVTAELKPNSRLCCQIIMTPELD
GIVVDVPDRQW

According to a further preferred embodiment of the present invention, the ferredoxin reductase used in the method according to the invention comprises an amino acid sequence which is at least 80%, preferably at least 85%, more preferably at least 90%, more preferably at least 95%, more preferably at least 96%, more preferably at least 97%, more preferably at least 98%, more preferably at least 99%, in particular 100%, identical to the amino acid sequence SEQ ID No. 8.

SEQ ID No. 8:
MNANDNVVIVGTGLAGVEVAFGLRASGWEGNIRLVGDATVIPHHLPPL
SKAYLAGKATAESLYLRTPDAYAAQNIQLLGGTQVTAINRDRQQVILS
DGRALDYDRLVLATGGRPRPLPVASGAVGKANNFRYLRTLEDAECIRR
QLIADNRLVVIGGGYIGLEVAATAIKANMHVTLLDTAARVLERVTAPP
VSAFYEHLHREAGVDIRTGTQVCGFEMSTDQQKVTAVLCEDGTRLPAD
LVIAGIGLIPNCELASAAGLQVDNGIVINEHMQTSDPLIMAVGDCARF
HSQLYDRWVRIESVPNALEQARKIAAILCGKVPRDEAAPWFWSDQYEI
GLKMVGLSEGYDRIIVRGSLAQPDFSVFYLQGDRVLAVDTVNRPVEFN
QSKQIITDRLPVEPNLLGDESVPLKEIIAAAKAELSSA

Amino acid sequences SEQ ID Nos. 7 and 8 are preferably encoded by nucleic acid sequences SEQ ID Nos. 9 and 10, respectively, with nucleic acid sequences SEQ ID Nos. 11 and 12 being optimized for expression in E. coli.

SEQ ID No. 9:
(ATG)_{0 or 1}
TCTAAAGTAGTGTATGTGTCACATGATGGAACGCGTCGCGAACTGGATG
TGGCGGATGGCGTCAGCCTGATGCAGGCTGCAGTCTCCAATGGTATCTA
CGATATTGTCGGTGATTGTGGCGGCAGCGCCAGCTGTGCCACCTGCCAT
GTCTATGTGAACGAAGCGTTCACGGACAAGGTGCCCGCCGCCAACGAGC
GGGAAATCGGCATGCTGGAGTGCGTCACGGCCGAACTGAAGCCGAACAG
CAGGCTCTGCTGCCAGATCATCATGACGCCCGAGCTGGATGGCATCGTG
GTCGATGTTCCCGATAGGCAATGGTAA
SEQ ID No. 10:
ATGAACGCAAACGACAACGTGGTCATCGTCGGTACCGGACTGGCTGGCG
TTGAGGTCGCCTTCGGCCTGCGCGCCAGCGGCTGGGAAGGCAATATCCG
GTTGGTGGGGGATGCGACGGTAATTCCCCATCACCTACCACCGCTATCC
AAAGCTTACTTGGCCGGCAAAGCCACAGCGGAAAGCCTGTACCTGAGAA
CCCCAGATGCCTATGCAGCGCAGAACATCCAACTACTCGGAGGCACACA
GGTAACGGCTATCAACCGCGACCGACAGCAAGTAATCCTATCGGATGGC
CGGGCACTGGATTACGACCGGCTGGTATTGGCTACCGGAGGGCGTCCAA
GACCCCTACCGGTGGCCAGTGGCGCAGTTGGAAAGGCGAACAACTTTCG
ATACCTGCGCACACTCGAGGACGCCGAGTGCATTCGCCGGCAGCTGATT
GCGGATAACCGTCTGGTGGTGATTGGTGGCGGCTACATTGGCCTTGAAG
TGGCTGCCACCGCCATCAAGGCGAACATGCACGTCACCCTGCTTGATAC
GGCAGCCCGGGTTCTGGAGCGGGTTACCGCCCCGCCGGTATCGGCCTTT
TACGAGCACCTACACCGCGAAGCCGGCGTTGACATACGAACCGGCACGC
AGGTGTGCGGGTTCGAGATGTCGACCGACCAACAGAAGGTTACTGCCGT
CCTCTGCGAGGACGGCACAAGGCTGCCAGCGGATCTGGTAATCGCCGGG
ATTGGCCTGATACCAAACTGCGAGTTGGCCAGTGCGGCCGGCCTGCAGG
TTGATAACGGCATCGTGATCAACGAACACATGCAGACCTCTGATCCCTT
GATCATGGCCGTCGGCGACTGTGCCCGATTTCACAGTCAGCTCTATGAC
CGCTGGGTGCGTATCGAATCGGTGCCCAATGCCTTGGAGCAGGCACGAA
AGATCGCCGCCATCCTCTGTGGCAAGGTGCCACGCGATGAGGCGGCGCC
CTGGTTCTGGTCCGATCAGTATGAGATCGGATTGAAGATGGTCGGACTG
TCCGAAGGGTACGACCGGATCATTGTCCGCGGCTCTTTGGCGCAACCCG
ACTTCAGCGTTTTCTACCTGCAGGGAGACCGGGTATTGGCGGTCGATAC
AGTGAACCGTCCAGTGGAGTTCAACCAGTCAAAACAAATAATCACGGAT
CGTTTGCCGGTTGAACCAAACCTACTCGGTGACGAAAGCGTGCCGTTAA
AGGAAATCATCGCCGCCGCCAAAGCTGAACTGAGTAGTGCCTGA
SEQ ID No. 11:
(ATG)_{0 or 1}
ATGAGCAAAGTGGTCTATGTGTCGCATGATGGAACACGCCGTGAGTTAG
ACGTCGCTGATGGTGTATCCCTGATGCAAGCAGCGGTTAGCAATGGCAT
TTACGACATCGTTGGCGATTGTGGTGGTAGTGCGTCATGTGCAACGTGT
CACGTGTATGTTAACGAAGCGTTTACCGATAAGGTGCCTGCTGCCAATG
AACGCGAGATTGGCATGCTGGAATGCGTAACTGCCGAACTCAAACCGAA
CTCTCGCCTGTGCTGCCAGATCATCATGACCCCGGAATTGGACGGGATT
GTCGTTGATGTGCCAGATCGTCAGTGGTAA
SEQ ID No. 12:
ATGAACGCCAATGATAATGTTGTTATTGTTGGCACCGGTCTGGCAGGCG
TTGAAGTTGCATTTGGTCTGCGTGCAAGCGGTTGGGAAGGTAATATTCG
TCTGGTTGGTGATGCAACCGTTATTCCGCATCATCTGCCTCCGCTGAGC
AAAGCATATCTGGCAGGTAAAGCAACCGCAGAAAGCCTGTATCTGCGTA
CACCGGATGCCTATGCAGCACAGAATATTCAGCTGCTGGGTGGTACACA
GGTTACCGCAATTAATCGTGATCGTCAGCAGGTTATTCTGAGTGATGGT
CGTGCACTGGATTATGATCGTCTGGTGCTGGCAACCGGTGGTCGTCCGC
GTCCGCTGCCGGTTGCAAGTGGTGCAGTTGGTAAAGCCAATAACTTTCG
TTATCTGCGCACCCTGGAAGATGCAGAATGTATTCGTCGTCAGCTGATT
GCAGATAATCGCCTGGTTGTGATTGGTGGTGGTTATATTGGTCTGGAAG
TTGCAGCAACCGCCATTAAAGCAAATATGCATGTTACCCTGCTGGATAC
CGCAGCACGTGTTCTGGAACGTGTTACCGCACCGCCTGTTAGCGCCTTT
TATGAACATCTGCATCGTGAAGCCGGTGTTCATATTCGTACCGGCACCC
AGGTTTGTGGTTTTGAAATGAGCACCGATCAGCAGAAAGTTACCGCAGT
TCTGTGTGAAGATGGCACCCGTCTGCCTGCAGATCTGGTTATTGCAGGT
ATTGGCCTGATTCCGAATTGTGAACTGGCAAGCGCAGCAGGTCTGGCAG
TTGGTGATTGTGCACGTTTTCATAGCCAGCTGTATGATCGTTGGGTTCG
TATTGAAAGCGTTCCGAATGCACTGGAACAGGCACGTAAAATTGCAGCA
ATTCTGTGTGGTAAAGTTCCGCGTGATGAAGCAGCACCGTGGTTTTGGA
GCGATCAGTATGAAATTGGTCTGAAAATGGTTGGTCTGAGCGAAGGTTA
TGATCGCATTATTGTTCGTGGTAGCCTGGCACAGCCGGATTTTTCAGTT
TTTTATCTGCAGGGTGATCGTGTGCTGGCAGTTGATACCGTTAATCGTC
CGGTTGAATTTAACCAGAGCAAACAAATTATCACCGATCGTCTGCCGGT
GGAACCGAATCTGCTGGGAGATGAAAGCGTGCCGCTGAAAGAAATTATT
GCAGCAGCAAAAGCAGAACTGAGCAGCGCATAA

The expression of the cytochrome P450 according to the invention and any ferredoxins and ferredoxin reductases in bacteria, in particular in E. coli, is particularly advantageous when nucleic acids with the nucleic acid sequences SEQ ID No. 5, SEQ ID No. 6, SEQ ID No. 11 and/or SEQ ID No. 12 are used. Further aspects of the present invention therefore relate to a nucleic acid (DNA and/or RNA) with a nucleic acid sequence selected from the group consisting of SEQ ID No. 5, SEQ ID No. 6, SEQ ID No. 11 and SEQ ID No. 12 and vectors and/or cells, in particular E. coli cells, comprising at least one of those sequences.

It has been shown that it is particularly advantageous if the above-mentioned ferredoxins and ferredoxin reductases are expressed (co-expressed) together with cytochrome P450 in a production strain (e.g., an E. coli strain). Through the co-expression of the three proteins or, respectively, enzymes, ideally under the same promoter, an ideal balance between the enzymes can be established, which has a particularly advantageous effect on the enzymatic conversion of a substrate.

According to a preferred embodiment of the present invention, the method according to the invention is performed at a temperature of from 10° to 40° C., preferably from 15° to 38° C., more preferably from 20° C. to 30° C., more preferably from 22° C. to 26° C. It has been shown according to the invention that the enzyme activity ofcytochrome P450 for the reaction according to the invention is particularly high in this area.

According to a further preferred embodiment of the present invention, the method according to the invention is performed at a pH offrom 6.5 to 8.5, preferably from 7 to 8, more preferably from 7.2 to 7.8. At this pH value, the enzyme activity of cytochrome P450 is highest so as to allow an appropriate conversion of the substrate.

The hydroxylation of deoxysteroids or, respectively, deoxysteroid derivatives can be carried out regioselectively at position 7 of the steroid backbone. In this way, in particular, a 7beta-hydroxyl group can be introduced stereoselectively so that, for example, ursocholic acid and/or ursocholic acid derivatives can be produced.

In the method according to the present invention, the isolation of the product can be effected in different ways. For example, the product can be extracted from the reaction mixture by a suitable organic solvent. Depending on the substrate, such solvents are described in the literature. According to the present invention, cholic acids and their derivatives can be isolated from reaction mixtures, for example, with ethyl acetate, optionally after acidification of the reaction mixture, e.g., with HCl. A method in which bile acids are present in the form of a salt, e.g., a sodium salt, in an aqueous solution constitutes a special case. In this case, a precipitation of the product can be effected by acidifying the reaction mixture. For this purpose, for example, HCl or dilute HCl can be added to the reaction mixture in a sufficient amount. If a pH value of, for example, 1 to 4, preferably 2 to 3, is achieved in the process, the product predominantly exists in the form of a suspension. The product can then be removed from the reaction mixture by common methods such as, e.g., filtration or centrifugation. Chromatographic methods, such as, e.g., column chromatography or flash chromatography, are another alternative that can be used for product isolation, for example. Furthermore, it is possible, for example, to obtain product by evaporating the reaction solvent.

Alternatively, in a method according to the present invention, the product(s) may also remain in the reaction mixture after the reaction, e.g., in order to carry out even more reactions and optionally isolate an end product upon completion of those reactions. It is also conceivable that the substrate(s) for the method according to the present invention is/are produced in the same reaction batch by previous reactions or reactions taking place in parallel.

### EXAMPLES

The present invention is explained in further detail using the following examples, without, however, being restricted thereto.

### Example 1: Test of Bacterial Strains

The following bacterial strains were obtained from the German Strain Collection of Microorganisms and Cell Cultures (DSMZ [Deutsche Stammsammlung für Mikroorganismen und Zellkulturen]): Saccharothrix longispora (DSM-43749), Catellatospora citrae (DSM-44097), Streptomyces hygroscopicus subsp. hygroscopicus (DSM-40578) and Asanoa ferruginea (DSM-44099). The strains were cultivated under standard conditions as recommended by DSMZ. As soon as the growth of the cultures had led to visible turbidity, deoxycholic acid (0.5 mM) was added, and it was cultured further for up to 72 h. After a centrifugation step, supernatants of the cultures were extracted with ethyl acetate and analyzed by HPLC and GC/MS. In the HPLC chromatogram of the reaction with Streptomyces hygroscopicus, a peak was noted the retention time of which corresponds to that of ursocholic acid. The GC/MS analysis indicated that the potential ursocholic acid peak originates from a bile acid with 3 hydroxyl groups. The examination of the other strains gave no indication of 7-hydroxylated products of deoxycholic acid.

### Example 2: Genome Sequencing and Annotation of the P450 Genes

Upon cultivation of Streptomyces hygroscopicus subsp. hygroscopicus (DSM-40578) according to the DSMZ regulation, the genomic DNA of the strain was isolated (Kieser et al. (2000), Practical Streptomyces genetics (Norwich: John Innes Foundation)). The genome was sequenced using Illumina MiSeq, and an assembly based on the known genome of Streptomyces rapamycinicus was conducted (Microsynth GmbH, Switzerland). 42 P450 genes could be identified by homology comparisons.

### Example 3: Cloning of Expression System

Using the restriction enzyme XhoI, the following construct comprising coding regions for putidaredoxin reductase (PtR) and putidaredoxin (Ptx) was cloned into plasmid pJ411 (DNA 2.0).

Synthetic DNA (Life Technologies): 5′, XhoI interface, HindIII interface, approx. 50 bp spacer DNA, ribosome binding site (rbs), ORF (open reading frame) putidaredoxin reductase (PtR), approx. 50 bp spacer DNA, rbs, ORF putidaredoxin (Ptx), XhoI interface, 3′.

The result of the cloning step was checked by means of restriction enzyme digestion and DNA sequencing.

Subsequently, using the restriction enzymes NdeI and HindIII, one ORF each coding for the P450 hydroxylases identified in Example 2 was cloned into the above-mentioned synthetic DNA or plasmid, respectively (Life Technologies). The result was again verified by means of restriction enzyme digestion and DNA sequencing. The expression vector used in this example and the redox partners used constitute only one way of expressing the cytochrome P450 enzymes according to the invention, which way has been chosen as an example.

The expression plasmids produced with the identified P450 candidates (see example 2) can be used for jointly expressing the respective P450 proteins together with putidaredoxin reductase and putidaredoxin. The 3 ORFs of the respective expression plasmids are expressed under the control of a T7 promoter on a common mRNA, but as separate polypeptides.

### Example 4: Expression of P450/Ptx/PtR

After the genome sequencing of Streptomyces hygroscopicus subsp. hygroscopicus, there were 42 P450 sequences that came into consideration as candidates for a possible deoxycholic acid-7-hydroxylase. To identify the enzyme looked for, ORFs of the candidates were cloned into the expression system described in example 3 and into a pJ411 (DNA 2.0) expression vector without coding regions for putidaredoxin reductase (PtR) and putidaredoxin (Ptx). The following protocol was used for the expression.

TB-P450 expression medium:
Terrific broth (TB) medium
+50 μg/ml kanamycin
+0.5 mM 5-aminolevulinic acid (from 100×parent solution)
+1 mM thiamine (from 100×parent solution)
+1 mM MgCl₂+2.5 mM ammonium sulfate+50 μM FeCl₃ (from 100×parent solution)
+0.5 mM IPTG (from 1 M parent solution)
(the additives were each 0.2 μm sterile filtered)

P450 lysis buffer:
100 mM Tris pH 7.5
20% (v/v) glycerin
1 mg/ml lysozyme

The constructs of the P450 candidates, which were to be tested, were transformed into the E. coli expression strain BL21 (DE3). Overnight cultures were inoculated from single colonies (LB (lysogeny broth)+kanamycin). The next day, 1:100 expression cultures were inoculated therewith (150 ml TB (terrific broth) -P450 expression medium) and were initially shaken at 370° C. in baffled flasks (1 L) for 3 h. Subsequently, the temperature was lowered to 240° C., and it was shaken for another 22 h. The cultures were harvested by centrifugation at 5000 g for 10 min, washed 1x with 0.9% (w/v) NaCl, and pellets were frozen at −80° C. The cell pellets were thawed, weighed and resuspended with an equivalent amount of P450 lysis buffer, incubated on ice for 1 h and then digested using a sonifier. Upon centrifugation (30 min, 21000 g), the supernatants were used for test reactions.

### Example 5: Testing of P450 Candidates for DA Hydroxylation

Reaction mixture:
10-80 μl 100 mM NADH (redox cofactor)
250 μl 1 M Tris-HCl pH 7.5
17.5 μl glycerin (50%)
100 μl 50 mM deoxycholic acid solution pH 8.5 (final 10 mM)
50 μl E. coli lysate P450/PtR/Ptx (see Example 4)
17.5-87.5 μl dH₂O

The reactions were set up in 1.5 ml screw-top bottles and sealed with lids with aluminium foil. The foil was punctured in several places. It was gently shaken at 24° C. for 18 h. 200 μl of the reaction batch was diluted with 600 μl acetonitrile/5 μl H₃PO₄ (50%) and incubated at 550° C. for 15 minutes. Subsequently, the samples were centrifuged at 20817 rcf for 5 minutes and analyzed using HPLC/DAD (e.g., Agilent 1200 series;
column: Merck Purospher STAR RP-18e 125×4 mm, 5 μm;
flow rate: 1.5 ml/min, gradient H₂O +H₃PO₄ (pH=2.6)/acetonitrile). One of the examined candidates (“P450_c866”) was able to hydroxylate deoxycholic acid to ursocholic acid. The deoxycholic acid used was converted in the process (see the following table). The identity of the product ursocholic acid was verified by GC/MS analysis and by 2D NMR.

| Redox cofactor [μl] | ursocholic acid [μg/ml] | conversion [%] |
|---|---|---|
| 10 | 30 | 3.4 |
| 30 | 48 | 5.3 |
| 50 | 57 | 6.2 |
| 80 | 110 | 11.6 |

In this example, a redox cofactor (NADH) is oxidized by the P450/Ptx/PtR reaction.

1. (canceled)

2. A method of preparing a steroid having the general formula (II): 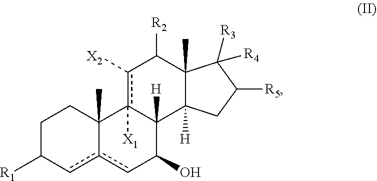
wherein
X₁ and X₂ are independently H, Cl, F, Br, I, CF₃, a C₁ to C₆ alkyl radical, OH, a C₁ to C₆ alkoxy radical, CN, NO₂, N(R₆)₂, an epoxy group, CHO, or a CO₂R₆ radical, wherein
R₆ is —C(O)H, —C(O)CH₃, —C(O)CH₂CH₃, —C(O)(CH₂)₂CH₃, —C(O)CH(CH₃)₂, —C(O)(CH₂)₃CH₃, —C(O)CH(CH₃)CH₂CH₃, —C(O)CH₂CH₂(CH₃)₂, —C(O)C(CH₃)₃, —C(O)Ph, or —C(O)CH₂Ph,
R₁ and R₂ are independently H, OH, OR₇, or O, wherein
R₇ is —C(O)H, —C(O)CH₃, —C(O)CH₂CH₃, —C(O)(CH₂)₂CH₃, —C(O)CH(CH₃)₂, —C(O)(CH₂)₃CH₃, —C(O)CH(CH₃)CH₂C₁₃, —C(O)CH₂CH-₂(CH₃)₂, —C(O)C(CH₃)₃, —C(O)Ph, or —C(O)CH₂Ph,
R₃ is H, OH, OR₈, a C₁ to C₁₀ alkyl radical, a C₁ to C₁₀ alkenyl radical, —CHO, —C(O)(CH₃), —C(O)(CH₂OH), —CH(CH₃)C(O)CH₃, —CH(CH₃)((CH₂)₂CO₂R₉), or —CH(CH₃)((CH₂)₂CONHR₉), wherein
R₈ is —C(O)H, —C(O)CH₃, —C(O)CH₂CH₃, —C(O)(CH₂)₂CH₃, —C(O)CH(CH₃)₂, —C(O)(CH₂)₃CH₃, —C(O)CH(CH₃)CH₂CH₃, —C(O)CH₂CH₂(CH₃)₂, —C(O)C(CH₃)₃, —C((O)Ph, or —C(O)CH₂Ph, and
R₉ is —CH₃, —CH₂COOH, —CH₂CH₃, —CH(CH₃)₂, —(CH₂)₂(C₃, —(CH₂)₂SO₃H, C(CH₃)₃, —(CH₂)₃CH₃, —CH(CH₃)CH₂CH₃, —CH₂CH₂(CH₃)₂, an aryl group, or an alkylaryl group,
R₄ is H, OH, or —OR₁₀, wherein
R₁₀ is —C(O)H, —C(O)CH₃, —C(O)CH₂CH₃, —C(O)(CH₂)₂CH₃, —C(O)CH(CH₃)₂, —C(O)(CH₂)₃CH₃, —C(O)CH(CH₃)CH₂CH₃, —C(O)CH₂CH₂(CH₃)₂, —C(O)C(CH₃)₃, —C(O)Ph, or —C(O)CH₂Ph, and
R₅ is H, CF₃, a C₁ to C₆ alkyl radical, a C₁ to C₆ alkenyl radical, OH, O, or a C₁ to C₆ alkoxy radical, wherein the dashed line denotes an optional double bond, with the proviso that the B ring has no double bond if the A ring has a C₄-C₅ double bond, and the C ring has no double bond if X₁ and X₂ form an epoxy group, or
wherein the steroid having the general formula (II) is selected from the group consisting of 3α,7β,12α-trihydroxy-5β-cholane-24-acid, 3α,7β,12α-trihydroxy-5β-cholane-24-acid, 3α,7β,12α-trihydroxy-5β-cholane-24-acid, 3β,7β,12α-trihydroxy-5β-cholane-24-acid, 7β,12α-dihydroxy-3-keto-5β-cholane-24-acid, 7β,12β-dihydroxy-3-keto-5β-cholane-24-acid, 3p,7β-diydroxy-12-keto-5β-cholane-24-acid, 3α,7β-dihydroxy-5β-cholane-24-acid, 7β-hydroxy-3-keto-5β-cholane-24-acid and 3β,7β-dihydroxy-5β-cholane-24-acid,
the method comprising the step of converting a 7-deoxysteroid selected from the group consisting of 3α,12α-dihydroxy-5β-cholane-24-acid, 3α,12β-dihydroxy-5β-cholane-24-acid, 3β,12α-dihydroxy-5β-cholane-24-acid, 3p,12β-dihydroxy-5β-cholane-24-acid, 3β-hydroxy-12-keto-5β-cholane-24-acid, 3-keto,12p-hydroxy-5β-cholane-24-acid, 3-keto,12α-hydroxy-5β-cholane-24-acid, 3α-hydroxy-5β-cholane-24-acid, 3-keto-5β-cholane-24-acid and 3β-hydroxy-5β-cholane-24-acid or having the general formula (I) with cytochrome P450 enzyme: 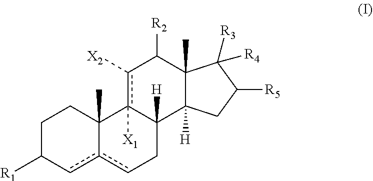
wherein the cytochrome P450 enzyme comprises an amino acid sequence which has an identity of at least 80% to the amino acid sequence of SEQ ID No. 1 or SEQ ID No. 2.

3. The method according to claim 2, wherein:
X₁, X₂, R₄ and R⁵ are H, and
R₁ und R₂ are independently H, OH, OR₇, or O, wherein
R₇_is —C(O)H, —C(O)CH₃, —C(O)CH₂CH₃, —C(O)(CH₂)₂CH₃, —C(O)CH(CH₃)₂, —C(O)(CH₂)₃CH₃, —C(C)CH(C H₃)C₁H₂CH₁₃ —C(O)CH₂CH(CH₃)₂, —C(O)C(CH₃)₃, —C(O)Ph, or —C(O)CH₂Ph, R₃ is a C₁ to C₁₀ alkyl radical, a C₁ to C₁₀ alkenyl radical, —CH(CH₃)((CH₂)₂CO₂R₉), or —CH(CH₃)((CH₂)₂CONHR₉), wherein
R₉ is —CH₃, —CH₂COOH, —CH₂CH₃, —CH(CH3)₂, —(CH₂)₂CH₃, —(CH₂)₂SO₃H—H, C(CH₃)₃, —(CH₂)₃CH₃, —CH(CH₃)CH₂CH₃, —CH₂CH(CH₃)₂, an aryl group, or an alkylaryl group.

4. The method according to claim 2, wherein the aryl group is selected from the group consisting of a phenyl radical, a phenyl radical substituted with F, Cl, Br, NO₂ or CH₃, and a heteroaryl.

5. The method according to claim 2, wherein the alkylaryl group is selected from the group consisting of a benzyl group, a halogenated benzyl group in which the halogen is F, C₁ or Br, and a benzyl group substituted with NO₂.

6. The method according to claim 2, wherein R₁ is OH, R₂ is O or OH, R₃ is —CH(CH₃)((CH₂)₂CO₂R₉), R₄ is H, and R₅ is H.

7. (canceled)

8. The method according to claim 2, wherein the enzymatic conversion takes place in the presence of at least one ferredoxin.

9. The method according to claim 2, wherein the enzymatic conversion takes place in the presence of at least one ferredoxin reductase.

10. The method according to claim 2, wherein the enzymatic conversion takes place in the presence of at least one ferredoxin and at least one ferredoxin reductase.

11. The method according to claim 2, wherein the cytochrome P450 enzyme comprises an amino acid sequence which has an identity of at least 90% to the amino acid sequence of SEQ ID No. 1 or SEQ ID No. 2.

12. The method according to claim 2, wherein the cytochrome P450 enzyme comprises an amino acid sequence which has an identity of 100% to the amino acid sequence of SEQ ID No. 1 or SEQ ID No. 2.


SEQUENCE LISTING (12 sequences)

SEQ ID NO: 1 - protein, 397 aa, Streptomyces hygroscopicus
MLTTAETTSIAYPFNTAEGLALSERYEEARNRTGLLRVRMPYGEPAWLVTRYADARLVLGDRRFSRAEALHHDEPRQSEGRRDSGILTMDPPDHTRLRTLVAKAFTVHQVEKLRPWVRQLTHDLLDDLEAAGPPADLVDRYALPIPVGVICAMLGVPQEDRPKFRVWSDAALSTSSLSAEQFARNTDELRAYMAGLIEDHRRTPRDDIMTSLIEARDAGDRLSELELVDLCVGILVAGHETTATQIPNFVLTLLEHPDQLRRLREDPALIQGAVEELLRFVPLGVGAAQARYATEDIEVGGTLVRSGEPVLVAVGSANRDALRFDEPGVLNVARPTTQHLGFGHGVHHCLGAPLARLELQEALGALITRFPGLRLAGDIEWKDRMLVRGPRVMPIGW

SEQ ID NO: 2 - protein, 358 aa, Streptomyces hygroscopicus
MPYGEPAWLVTRYADARLVLGDRRFSRAEALHHDEPRQSEGRRDSGILTMDPPDHTRLRTLVAKAFTVHQVEKLRPWVRQLTHDLLDDLEAAGPPADLVDRYALPIPVGVICAMLGVPQEDRPKFRVWSDAALSTSSLSAEQFARNTDELRAYMAGLIEDHRRTPRDDIMTSLIEARDAGDRLSELELVDLCVGILVAGHETTATQIPNFVLTLLEHPDQLRRLREDPALIQGAVEELLRFVPLGVGAAQARYATEDIEVGGTLVRSGEPVLVAVGSANRDALRFDEPGVLNVARPTTQHLGFGHGVHHCLGAPLARLELQEALGALITRFPGLRLAGDIEWKDRMLVRGPRVMPIGW

SEQ ID NO: 3 - DNA, 1194 bp, Streptomyces hygroscopicus
atgttgaccacagccgagacgacatccatcgcctatcccttcaacaccgccgaagggctggcgctcagcgagcgttacgaagaggccaggaaccgcaccggactgctccgggtgcggatgccctacggtgagcccgcctggctggtcacgcggtacgccgacgcccggctggtgctcggcgaccggcgcttcagccgtgcggaggcgctccaccacgacgagccgcggcagtccgaaggccggcgcgacagcggcatcctgaccatggacccgcccgaccacacccggctgcgcaccctcgtcgccaaggcgttcaccgtccaccaggtggagaaactccgcccctgggtacgccagttgacccatgacctgctcgacgacctcgaggccgccgggccgcccgccgatctggtggaccgctacgccctgcccattccggtcggcgtcatctgcgccatgctcggcgtcccgcaggaggaccggcccaagttccgggtctggagcgacgccgcgctgtccaccagctcgctgagcgccgagcagttcgcccgtaacaccgacgagctgcgcgcctacatggccgggctgatcgaggaccaccgcaggaccccgcgggacgacatcatgacctcgctgatcgaggcgcgggacgcgggcgaccggctgtccgagctggaactcgtcgatctgtgcgtgggcatcctggtggccgggcacgagaccaccgccacccagatccccaacttcgtgctgacgctgctggagcacccggaccagctgcgccggctgcgcgaggaccccgccctgatccagggcgccgtcgaggagctgctgcgcttcgtcccgctgggcgtgggcgccgcccaggcccgttacgccaccgaggacatcgaggtgggcggcacgctggtgcgcagcggggagccggtgctggtcgccgtcggctcggccaaccgcgacgcgctgcgcttcgacgaaccgggcgtgctcaacgtcgcccgccccaccacccagcacctcggcttcggccacggtgtgcaccactgcctgggcgcgcccctggcccgtctggagctccaggaggcgctcggcgcgctgatcacgcgcttcccgggcctgcggctggccggggacatcgagtggaaggaccgcatgctggtccgcgggccccgtgtcatgcccatcgggtggtga

SEQ ID NO: 4 - DNA, 1077 bp, Streptomyces hygroscopicus
atgccctacggtgagcccgcctggctggtcacgcggtacgccgacgcccggctggtgctcggcgaccggcgcttcagccgtgcggaggcgctccaccacgacgagccgcggcagtccgaaggccggcgcgacagcggcatcctgaccatggacccgcccgaccacacccggctgcgcaccctcgtcgccaaggcgttcaccgtccaccaggtggagaaactccgcccctgggtacgccagttgacccatgacctgctcgacgacctcgaggccgccgggccgcccgccgatctggtggaccgctacgccctgcccattccggtcggcgtcatctgcgccatgctcggcgtcccgcaggaggaccggcccaagttccgggtctggagcgacgccgcgctgtccaccagctcgctgagcgccgagcagttcgcccgtaacaccgacgagctgcgcgcctacatggccgggctgatcgaggaccaccgcaggaccccgcgggacgacatcatgacctcgctgatcgaggcgcgggacgcgggcgaccggctgtccgagctggaactcgtcgatctgtgcgtgggcatcctggtggccgggcacgagaccaccgccacccagatccccaacttcgtgctgacgctgctggagcacccggaccagctgcgccggctgcgcgaggaccccgccctgatccagggcgccgtcgaggagctgctgcgcttcgtcccgctgggcgtgggcgccgcccaggcccgttacgccaccgaggacatcgaggtgggcggcacgctggtgcgcagcggggagccggtgctggtcgccgtcggctcggccaaccgcgacgcgctgcgcttcgacgaaccgggcgtgctcaacgtcgcccgccccaccacccagcacctcggcttcggccacggtgtgcaccactgcctgggcgcgcccctggcccgtctggagctccaggaggcgctcggcgcgctgatcacgcgcttcccgggcctgcggctggccggggacatcgagtggaaggaccgcatgctggtccgcgggccccgtgtcatgcccatcgggtggtga

SEQ ID NO: 5 - DNA, 1194 bp, Artificial sequence
  E.coli codon-optimized SEQ ID No. 3
atgctgaccaccgcagaaaccaccagtattgcatatccgtttaataccgcagaaggtctggcactgagcgaacgttatgaagaagcacgtaatcgtaccggtctgctgcgtgttcgtatgccgtatggtgaaccggcatggctggttacccgttatgcagatgcccgtctggttctgggtgatcgtcgttttagccgtgccgaagcactgcatcacgatgaaccgcgtcagagcgaaggtcgtcgtgatagcggtattctgaccatggatccgcctgatcatacccgtctgcgtaccctggttgcaaaagcatttaccgttcatcaggttgaaaaactgcgtccgtgggttcgccagctgacccatgatctgctggatgatctggaagcagcaggtccgcctgcagatctggttgatcgttatgcactgccgattccggttggtgttatttgtgcaatgctgggtgttccgcaagaagatcgtcctaaatttcgtgtttggagtgatgcagcactgagcaccagcagcctgagcgcagaacagtttgcacgtaataccgatgaactgcgtgcatatatggcaggtctgattgaagatcatcgtcgtacaccgcgtgatgatattatgaccagcctgatcgaagcacgtgatgccggtgatcgcctgagtgaactggaactggtggatctgtgtgttggtattctggttgcaggtcatgaaaccaccgcaacccagattccgaattttgttctgaccctgctggaacatccggatcagctgcgtcgtctgcgtgaagatccggcactgattcagggtgcagttgaagaactgctgcgttttgttccgctgggtgtgggtgcagcacaggcacgttatgcaaccgaagatattgaagttggtggcaccctggttcgtagtggcgaaccggtgctggttgccgttggtagcgcaaaccgtgatgcactgcgctttgatgaaccgggtgttctgaatgttgcacgtccgaccacacagcatctgggttttggtcatggtgttcatcattgtctgggtgcaccgctggcacgtctggaactgcaagaagcactgggagcactgattacccgttttccgggtctgcgtctggcaggcgatattgaatggaaagatcgtatgctggttcgtggtccgcgtgttatgccgattggttggtaa

SEQ ID NO: 6 - DNA, 1070 bp, Artificial sequence
  E.coli codon-optimized SEQ ID No. 4
atggtgaaccggcatggctggttacccgttatgcagatgcccgtctggttctgggtgatcgtcgttttagccgtgccgaagcactgcatcacgatgaaccgcgtcagagcgaaggtcgtcgtgatagcggtattctgaccatggatccgcctgatcatacccgtctgcgtaccctggttgcaaaagcatttaccgttcatcaggttgaaaaactgcgtccgtgggttcgccagctgacccatgatctgctggatgatctggaagcagcaggtccgcctgcagatctggttgatcgttatgcactgccgattccggttggtgttatttgtgcaatgctgggtgttccgcaagaagatcgtcctaaatttcgtgtttggagtgatgcagcactgagcaccagcagcctgagcgcagaacagtttgcacgtaataccgatgaactgcgtgcatatatggcaggtctgattgaagatcatcgtcgtacaccgcgtgatgatattatgaccagcctgatcgaagcacgtgatgccggtgatcgcctgagtgaactggaactggtggatctgtgtgttggtattctggttgcaggtcatgaaaccaccgcaacccagattccgaattttgttctgaccctgctggaacatccggatcagctgcgtcgtctgcgtgaagatccggcactgattcagggtgcagttgaagaactgctgcgttttgttccgctgggtgtgggtgcagcacaggcacgttatgcaaccgaagatattgaagttggtggcaccctggttcgtagtggcgaaccggtgctggttgccgttggtagcgcaaaccgtgatgcactgcgctttgatgaaccgggtgttctgaatgttgcacgtccgaccacacagcatctgggttttggtcatggtgttcatcattgtctgggtgcaccgctggcacgtctggaactgcaagaagcactgggagcactgattacccgttttccgggtctgcgtctggcaggcgatattgaatggaaagatcgtatgctggttcgtggtccgcgtgttatgccgattggttggtaa

SEQ ID NO: 7 - protein, 107 aa, Pseudomonas putida
  MISC_FEATURE (1)..(1): Xaa is methionine or no amino acid residue
XSKVVYVSHDGTRRELDVADGVSLMQAAVSNGIYDIVGDCGGSASCATCHVYVNEAFTDKVPAANEREIGMLECVTAELKPNSRLCCQIIMTPELDGIVVDVPDRQW

SEQ ID NO: 8 - protein, 422 aa, Pseudomonas putida
MNANDNVVIVGTGLAGVEVAFGLRASGWEGNIRLVGDATVIPHHLPPLSKAYLAGKATAESLYLRTPDAYAAQNIQLLGGTQVTAINRDRQQVILSDGRALDYDRLVLATGGRPRPLPVASGAVGKANNFRYLRTLEDAECIRRQLIADNRLVVIGGGYIGLEVAATAIKANMHVTLLDTAARVLERVTAPPVSAFYEHLHREAGVDIRTGTQVCGFEMSTDQQKVTAVLCEDGTRLPADLVIAGIGLIPNCELASAAGLQVDNGIVINEHMQTSDPLIMAVGDCARFHSQLYDRWVRIESVPNALEQARKIAAILCGKVPRDEAAPWFWSDQYEIGLKMVGLSEGYDRIIVRGSLAQPDFSVFYLQGDRVLAVDTVNRPVEFNQSKQIITDRLPVEPNLLGDESVPLKEIIAAAKAELSSA

SEQ ID NO: 9 - DNA, 324 bp, Pseudomonas putida
  misc_feature (1)..(1): n is A or no nucleotide
  misc_feature (2)..(2): n is T or no nucleotide
  misc_feature (3)..(3): n is G or no nucleotide
nnntctaaagtagtgtatgtgtcacatgatggaacgcgtcgcgaactggatgtggcggatggcgtcagcctgatgcaggctgcagtctccaatggtatctacgatattgtcggtgattgtggcggcagcgccagctgtgccacctgccatgtctatgtgaacgaagcgttcacggacaaggtgcccgccgccaacgagcgggaaatcggcatgctggagtgcgtcacggccgaactgaagccgaacagcaggctctgctgccagatcatcatgacgcccgagctggatggcatcgtggtcgatgttcccgataggcaatggtaa

SEQ ID NO: 10 - DNA, 1269 bp, Pseudomonas putida
atgaacgcaaacgacaacgtggtcatcgtcggtaccggactggctggcgttgaggtcgccttcggcctgcgcgccagcggctgggaaggcaatatccggttggtgggggatgcgacggtaattccccatcacctaccaccgctatccaaagcttacttggccggcaaagccacagcggaaagcctgtacctgagaaccccagatgcctatgcagcgcagaacatccaactactcggaggcacacaggtaacggctatcaaccgcgaccgacagcaagtaatcctatcggatggccgggcactggattacgaccggctggtattggctaccggagggcgtccaagacccctaccggtggccagtggcgcagttggaaaggcgaacaactttcgatacctgcgcacactcgaggacgccgagtgcattcgccggcagctgattgcggataaccgtctggtggtgattggtggcggctacattggccttgaagtggctgccaccgccatcaaggcgaacatgcacgtcaccctgcttgatacggcagcccgggttctggagcgggttaccgccccgccggtatcggccttttacgagcacctacaccgcgaagccggcgttgacatacgaaccggcacgcaggtgtgcgggttcgagatgtcgaccgaccaacagaaggttactgccgtcctctgcgaggacggcacaaggctgccagcggatctggtaatcgccgggattggcctgataccaaactgcgagttggccagtgcggccggcctgcaggttgataacggcatcgtgatcaacgaacacatgcagacctctgatcccttgatcatggccgtcggcgactgtgcccgatttcacagtcagctctatgaccgctgggtgcgtatcgaatcggtgcccaatgccttggagcaggcacgaaagatcgccgccatcctctgtggcaaggtgccacgcgatgaggcggcgccctggttctggtccgatcagtatgagatcggattgaagatggtcggactgtccgaagggtacgaccggatcattgtccgcggctctttggcgcaacccgacttcagcgttttctacctgcagggagaccgggtattggcggtcgatacagtgaaccgtccagtggagttcaaccagtcaaaacaaataatcacggatcgtttgccggttgaaccaaacctactcggtgacgaaagcgtgccgttaaaggaaatcatcgccgccgccaaagctgaactgagtagtgcctga

SEQ ID NO: 11 - DNA, 327 bp, Artificial sequence
  E.coli codon-optimized SEQ ID No. 9
  misc_feature (1)..(1): n is A or no nucleotide
  misc_feature (2)..(2): n is T or no nucleotide
  misc_feature (3)..(3): n is G or no nucleotide
nnnatgagcaaagtggtctatgtgtcgcatgatggaacacgccgtgagttagacgtcgctgatggtgtatccctgatgcaagcagcggttagcaatggcatttacgacatcgttggcgattgtggtggtagtgcgtcatgtgcaacgtgtcacgtgtatgttaacgaagcgtttaccgataaggtgcctgctgccaatgaacgcgagattggcatgctggaatgcgtaactgccgaactcaaaccgaactctcgcctgtgctgccagatcatcatgaccccggaattggacgggattgtcgttgatgtgccagatcgtcagtggtaa

SEQ ID NO: 12 - DNA, 1269 bp, Artificial sequence
  E.coli codon-optimized SEQ ID No. 10
atgaacgccaatgataatgttgttattgttggcaccggtctggcaggcgttgaagttgcatttggtctgcgtgcaagcggttgggaaggtaatattcgtctggttggtgatgcaaccgttattccgcatcatctgcctccgctgagcaaagcatatctggcaggtaaagcaaccgcagaaagcctgtatctgcgtacaccggatgcctatgcagcacagaatattcagctgctgggtggtacacaggttaccgcaattaatcgtgatcgtcagcaggttattctgagtgatggtcgtgcactggattatgatcgtctggtgctggcaaccggtggtcgtccgcgtccgctgccggttgcaagtggtgcagttggtaaagccaataactttcgttatctgcgcaccctggaagatgcagaatgtattcgtcgtcagctgattgcagataatcgcctggttgtgattggtggtggttatattggtctggaagttgcagcaaccgccattaaagcaaatatgcatgttaccctgctggataccgcagcacgtgttctggaacgtgttaccgcaccgcctgttagcgccttttatgaacatctgcatcgtgaagccggtgttgatattcgtaccggcacccaggtttgtggttttgaaatgagcaccgatcagcagaaagttaccgcagttctgtgtgaagatggcacccgtctgcctgcagatctggttattgcaggtattggcctgattccgaattgtgaactggcaagcgcagcaggtctgcaggttgataatggtattgttattaacgaacacatgcagaccagcgatccgctgattatggcagttggtgattgtgcacgttttcatagccagctgtatgatcgttgggttcgtattgaaagcgttccgaatgcactggaacaggcacgtaaaattgcagcaattctgtgtggtaaagttccgcgtgatgaagcagcaccgtggttttggagcgatcagtatgaaattggtctgaaaatggttggtctgagcgaaggttatgatcgcattattgttcgtggtagcctggcacagccggatttttcagttttttatctgcagggtgatcgtgtgctggcagttgataccgttaatcgtccggttgaatttaaccagagcaaacaaattatcaccgatcgtctgccggtggaaccgaatctgctgggagatgaaagcgtgccgctgaaagaaattattgcagcagcaaaagcagaactgagcagcgcataa